(19) United States
(12) Patent Application Publication
(10) Pub. No.: US 2025/0229025 A1
(43) Pub. Date: Jul. 17, 2025

(54) MULTI-CARTRIDGE INJECTOR AND METHODS OF MAKING AND USING SAME

(71) Applicant: Merck Sharp & Dohme LLC, Rahway, NJ (US)

(72) Inventors: Eduardo Cristofolli, Rahway, NJ (US); Cassie Megna, Rahway, NJ (US); Wail A. Rasheed, Rahway, NJ (US); Ophelia L. Wells, Easton, PA (US)

(73) Assignee: Merck Sharp & Dohme LLC, Rahway, NJ (US)

(21) Appl. No.: 18/701,430

(22) PCT Filed: Nov. 1, 2022
(86) PCT No.: PCT/US2022/048557

§ 371 Date: Apr. 15, 2024

Related U.S. Application Data
(60) Provisional application No. 63/275,099, filed on Nov. 3, 2021.

(51) Int. Cl. A61M 5/19; A61M 5/31; A61M 5/315; A61M 5/32

(52) U.S. Cl. CPC A61M 5/19; A61M 5/31505; A61M 5/31596; A61M 5/3294; A61M 2005/3103

(57) ABSTRACT

A multi-cartridge injector includes a plunger having a main brace and a pair of stems, each of the pair of stems having a coupler, a pair of stoppers coupled to each coupler of the pair of stems, a containment unit having a pair of barrels, and a Y-shaped conduit having a pair of branches and a common shaft, and a passageway that extends through the common shaft and the pair of branches, each of the pair of branches being in communication with an interior of one of the pair of barrels.

### FIELD OF THE DISCLOSURE

The present disclosure relates generally to syringes and injectors. More specifically, the present disclosure relates to multi-cartridge injectors.

### BACKGROUND OF THE DISCLOSURE

Prefilled syringes and cartridges are highly accurate parenteral devices used for the delivery of vaccines, biologics, therapies, and the like. Parenteral products contain many advantages such as minimizing drug waste and increasing product life span. Additionally, parenteral devices are easily recognized by healthcare workers as a convenient method of drug delivery and require minimal training to administer.

Pen injectors, autoinjectors, and cartridges are easy-to-use, accurate delivery devices used by many clinics and hospitals for delivery of vaccines, biologics, and therapies. These devices are easily recognized by healthcare workers as a convenient method of drug delivery. Pen and autoinjectors are mainly used for subcutaneous or intramuscular delivery of biopharmaceuticals and are focused on self-administration by the patient. However, conventional delivery devices in clinics are constrained and/or limited to one single drug product formulation to be delivered at the time of injection. Custom vials or cartridges require expensive manufacturing procedures, unknown interaction of the drug products, and increased risk for a dual-drug combined delivery. Additionally, conventional devices may result in a shorter shelf-life than desired, or in the need of multiple injections.

Thus, there exists a need for devices that improve upon and advance the methods of safely delivering combination products.

### SUMMARY OF THE DISCLOSURE

In some embodiments, a multi-cartridge injector includes a plunger having a main brace and a pair of stems, each of the pair of stems having a coupler, a pair of stoppers coupled to each coupler of the pair of stems, a containment unit having a pair of barrels, and a Y-shaped conduit having a pair of branches and a common shaft, the Y-shaped conduit defining a passageway that extends through the common shaft and the pair of branches, each of the pair of branches being in communication with an interior of one of the pair of barrels.

In some embodiments, a multi-cartridge injector includes a containment unit having a pair of barrels, and a Y-shaped conduit having a pair of branches and a common shaft, the Y-shaped conduit defining a passageway that extends through the common shaft and the pair of branches, each of the pair of branches being in communication with an interior of one of the pair of barrels.

### BRIEF DESCRIPTION OF THE DISCLOSURE

Various embodiments of the presently disclosed injectors are disclosed herein with reference to the drawings, wherein:

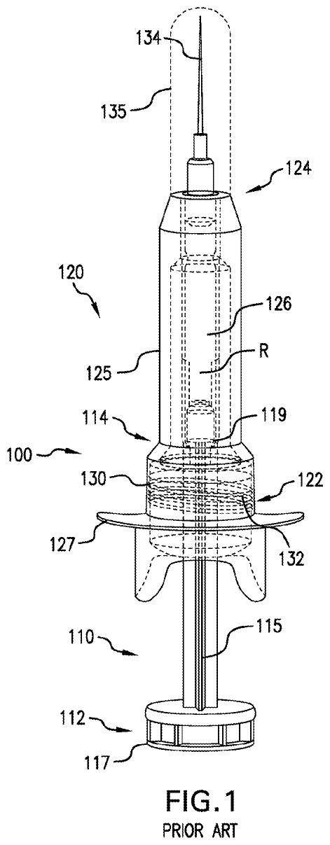

FIG. 1 is a schematic front view of a conventional pre-filled syringe with a safety device:

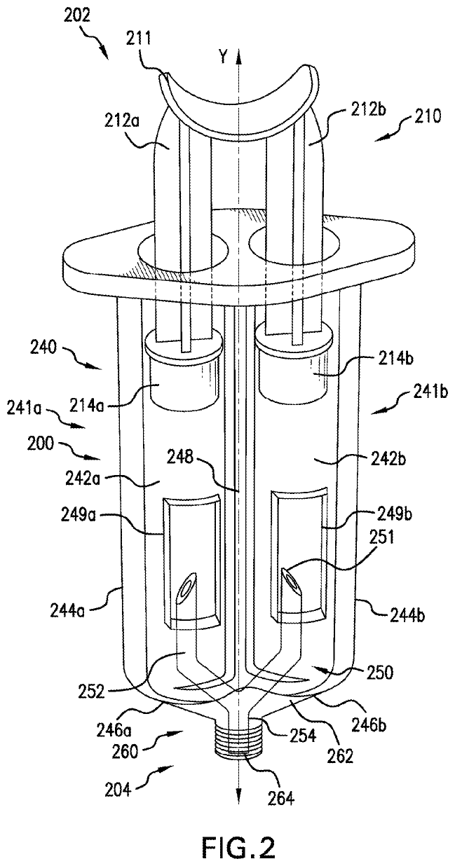

FIG. 2 is a schematic perspective view of a dual cartridge pen injector according to one embodiment of the disclosure:

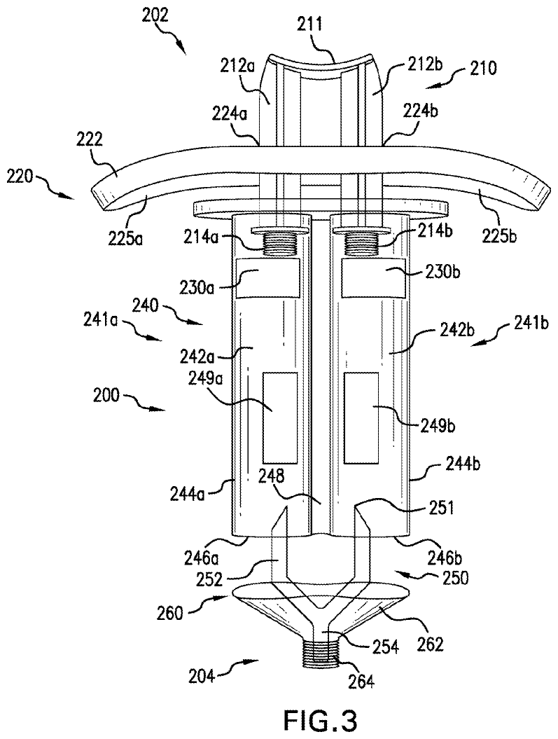

FIG. 3 is a schematic front view of the dual cartridge pen injector having an optional flange:

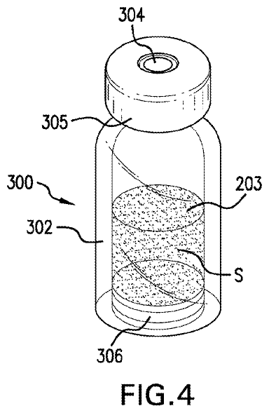

FIG. 4 is a schematic perspective view of a container closure system.

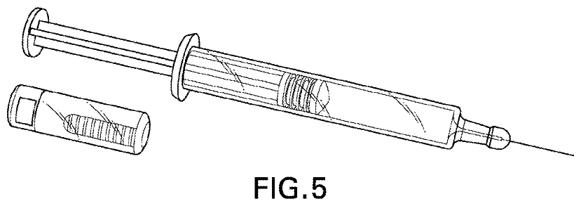

FIG. 5 is an image of a naked staked needle syringe; and

FIG. 6 is a schematic front view of a conventional pre-filled syringe.

Various embodiments will now be described with reference to the appended drawings. It is to be appreciated that these drawings depict only some embodiments of the disclosure and are therefore not to be considered limiting of its scope.

### DETAILED DESCRIPTION

Despite the various improvements that have been made to injectors and syringes, such as pre-filled syringes, conventional methods suffer from some shortcomings as discussed above.

Therefore, there is a need for further improvements to the devices and methods used to deliver medication. Among other advantages, the present disclosure may address one or more of these needs.

As used herein, the term “proximal,” when used in connection with a component of a syringe or injector, refers to the end of the component closest to the user's hands when holding the device, whereas the term “distal,” when used in connection with a component of a syringe or injector, refers to the end of the component closest to the needle insertion site during use.

Likewise, the terms “trailing” and “leading” are to be taken as relative to the operator's fingers (e.g., physician) of the syringe or injector. “Trailing” is to be understood as relatively close to the operator's fingers, and “leading” is to be understood as relatively farther away from the operator's fingers.

FIG. 1 shows a pre-filled syringe with a safety device and FIG. 6 shows a pre-filled syringe without a safety device. It is understood that FIG. 1 with the safety device is illustrative of a single cartridge injector and while this safety device is not applicable to a multi-cartridge injector (as exemplified in FIG. 2), a similar exemplary safety device could be applicable to a multi-cartridge injector.

Reference is now made to FIG. 1, which shows an exemplary prefilled-syringe 100 contained within a needle safety device having two states, a first state with the needle extended before injection (FIG. 1), and a second state with the needle retracted within a barrel after the full injection has been completed. It will be understood that though a needle within a safety device is shown, the disclosure is not thus limited. A naked staked needle syringe is also exemplified in FIGS. 5, 6 and 7.

Sensors of the present disclosure may be integrated into a specially designed syringe barrel or through the use of a separate assembly that could be attached to the syringe prior to use. Additionally, though a pre-filled syringe with a staked needle is shown, it will be understood that the principles disclosed herein are equally applicable to other types of injectors (e.g., syringes with removable needles, auto-injectors, or on-body (wearable) injectors, etc.).

Pre-filled syringe 100 generally comprises two main portions, a plunger rod 110 and a barrel 120, also represented in FIG. 6 as 400 and 401, respectively. Plunger rod 110 generally extends between a proximal end 112 and a distal end 114 and comprises an elongated piston 115 extending between a plunger flange 117 and a coupler 119. In one embodiment, piston 115 has a cruciform cross-sectional shape.

In FIG. 1, a cylindrical barrel 120 extends between proximal end 122 and distal end 124 and comprises a body 125 defining a lumen 126 for accepting a portion of plunger rod 110. Body 125 further comprises a barrel flange 127 adjacent proximal end 122 and defines a reservoir “R” that holds a medicament, drug, saline, or other substance for injecting into a patient's body. An internally threaded stopper 130 is disposed inside lumen 126 of body 125. In one embodiment, stopper 130 is made of an elastomeric material such as natural rubber, synthetic rubber, thermoplastic elastomers, or combinations thereof, and comprises an opening to receive and mate with coupler 119 of plunger rod 110 by advancing the plunger rod inside the barrel lumen 126 and rotating at least one of coupler 119 and stopper 130 relative to the other.

In FIG. 1, pre-filled syringe 100 includes a spring 132 operatively coupled to needle 134 to provide an additional safety mechanism. A cap 135 is also disposed over needle 134. Once cap 135 is removed, the user may pierce the patient's skin with the needle, then push on plunger flange 117 to drive the plunger to deliver a medicament through needle 134 into the patient's body. Spring 130 is configured so that, upon actuation and full delivery of the medicament, needle 134 will safely retract within barrel 120 and be locked inside to reduce the risk of needlestick injuries. It will be understood that while this configuration is limited to delivering a single produce from reservoir “R”, that some of the principles and components disclosed may be used in connection with multi-cartridge embodiments of the present disclosure.

Reference is now made to FIG. 6, which shows an exemplary pre-filled syringe 400 having with the needle extended before injection (FIG. 6).

Sensors of the present disclosure may be integrated into a specially designed syringe barrel or through the use of a separate assembly that could be attached to the syringe prior to use. Additionally, though a pre-filled syringe with a staked needle is shown, it will be understood that the principles disclosed herein are equally applicable to other types of injectors (e.g., syringes with removable needles, auto-injectors, or on-body (wearable) injectors, etc.).

Pre-filled syringe 400 generally comprises two main portions, a plunger rod 400 and a barrel 401. Plunger rod 400 generally extends between a proximal end 402 and a distal end 403 and comprises an elongated piston 404 extending between a plunger flange and a coupler. In one embodiment, piston 404 has a cruciform cross-sectional shape.

In FIG. 6, a cylindrical barrel 401 extends between proximal end and distal end and comprises a body 405 defining a lumen 406 for accepting a portion of plunger rod 400. Body 405 further comprises a barrel flange adjacent proximal end and defines a reservoir “R” that holds the medicament, drug, saline, or other substance for injecting into a patient's body. An internally threaded stopper is disposed inside lumen 406 of body 405. In one embodiment, stopper is made of an elastomeric material such as natural rubber, synthetic rubber, thermoplastic elastomers, or combinations thereof, and comprises an opening to receive and mate with coupler of plunger rod by advancing the plunger rod inside the barrel lumen 406 and rotating at least one of coupler and stopper relative to the other.

In FIG. 6, A cap 408 is also disposed over needle 407. Once cap 408 is removed, the user may pierce the patient's skin with the needle, then push on plunger flange 409 to drive the plunger to deliver a medicament through needle 407 into the patient's body. It will be understood that while this configuration is limited to delivering a single produce from reservoir “R”, that some of the principles and components disclosed may be used in connection with multi-cartridge embodiments of the present disclosure.

In another embodiment, the syringe is a naked staked needle syringe without a safety device, as shown in FIG. 5 and FIG. 6.

In some embodiments, a dual-chamber or dual cartridge injector may allow for the simultaneous delivery of the contents of two separate container closure systems (CCS) into a single injection site. In some embodiments, a multi-cartridge pen injector holds and injects solutions or products from two separate cartridges from a common or shared needle.

Turning to FIGS. 2-3, an exemplary multi-cartridge injector 200 includes a pair of barrels or cartridges. Injector 200 is configured to be generally symmetrical about a longitudinal central axis “Y”. Injector 200 generally extends between a proximal end 202 and a distal end 204. Moving from the proximal end to the distal end, injector 200 includes a plunger 210 that is formed of a main brace 211, and a pair of stems 212a,212b that terminate in couplers 214a,214b, respectively. Main brace 211 may be generally concave and shaped to receive a thumb or other digit of the user. Main brace 211 may connect to stems and each of stems 212a,212b may have a cruciform cross-sectional shape. Opposite main brace 211, couplers 214a,214b may be connected to a pair of stoppers 230a,230b. An optional flange 220 (shown only in FIG. 3) may be disposed adjacent or about plunger 210. In one example, flange 220 includes a body 222 that defines a pair of apertures 224a,224b, each of the apertures being sized and spaced to receive one of the stems 212a,212b, the stems being moveable relative to the body 222. In some examples, flange 220 is unitarily formed with the containment unit. In the example shown, body 222 is curved so that a user may place two digits on the underneath surfaces 225a,225b of the body 222 while pressing on main brace 211 to deliver the contents of the injector. In an alternative embodiment, flange 220) is not provided and enlarged shoulder 225 of the containment device may be used to serve a similar function.

A pair of internally threaded stoppers 230a,240b are connected to couplers 214a,214b. In one embodiment, stoppers 230a,240b are made of an elastomeric material such as natural rubber, synthetic rubber, thermoplastic elastomers, or combinations thereof, and comprise an opening to receive and mate with couplers 214a,214b of plunger 210.

Injector 200 further includes a containment unit 240 that includes a pair of barrels 241a,241b. In some examples, each of barrels 241a,241b is substantially cylindrically shaped and each includes a sidewall 244a,244b and a respective lumen 242a,242b for accepting at least a portion of stems 224a,224b and stoppers 230a,230b. Barrels 241a,241b may terminate in openings 246a,246b close to the distal end 204. In some examples, the two barrels 241a,241b are disposed side-by-side and may be joined at a common wall 248. Optionally, each of barrels 241a,241b may include a respective rectangular window 249a,249b, and the window may be sized to receive the vials or container closure systems, such as those of FIG. 4. In other examples, the vials or container closure systems are loaded from a bottom of a barrel and the windows 249a,249b are transparent or open cutouts used as a visualization tool to see inside the containment unit. The window allows the user to see the plunger cover the window to verify when the injector is completed and the drug product is dispensed. The window also allows the user as a visual aid to identify the vial content, including but not limited to particulate, color, defects, etc).

The containment unit may be used to hold the two separate container closure systems during packaging and shipping. Specifically, each barrel 241a,241b may define a respective reservoir R1,R2 that holds a medicament, drug, saline, or other substance for injecting into a patient's body. In some examples, reservoirs R1,R2 are sized to accept a container closure system, custom vial, or cartridge and these terms are used interchangeably throughout the disclosure. Preferably, reservoirs R1, R2 are sized to accept a cartridge, where the cartridge has a movable elastomeric stopper (306) and a septum to pierce. Another example would be a custom vial that is similar to a cartridge. A standard vial is unlikely to work due to the geometry or access points.

One example of such a cartridge or custom vial is shown in FIG. 4. Vaccines and biologics may be stored in cartridge or custom vial 300 made of borosilicate glass, known for its chemical stability and its ability to withstand long refrigeration and impact during transportation. Additionally, borosilicate glass has a low likelihood of chemical leaching. The cartridge or custom vials 300 may include a glass body 302 and a movable elastomeric stopper 306, disposed on one end and translatable within the glass body 302. Body 302 itself defines a main chamber 303 for receiving a substance “S” or medicament (e.g., vaccine, biologic, therapeutic, drug product for use in early drug development phases, etc.), and a latex septum 304 having a circumferential aluminum crimp 305 disposed on an opposite end. The cartridge or custom vial is not limited to those described or shown in FIG. 4 but must contain a septum (304) and movable elastomer stopper (306).

Turning back to FIG. 3, a Y-shaped conduit 250 may be used with injector 200. Y-shaped conduit 250 may include a pair of branches 252, each having a piercing tip 251, the branches meeting and joining at a common hollow shaft 254. Y-shaped conduit may define a continuous lumen from each of the branches 252 down to the common hollow shaft, and the tips of the two branches 252 may be sized and spaced so that they pierce a container within each of the barrels 241a,241b.

Y-shaped conduit 250 may be partially disposed within each of barrels 241a,241b, and common hollow shaft 254 may be at least partially disposed within cap 260. Cap 260 may be funnel-shaped at a first end 262 and have a luer-fitting 264 at an opposite second end configured and arranged to mate with a disposable probe needle (e.g., 18-gauge disposable needle). Thus, a standard off-the-shelf luer lock probe needle may engage with the luer-lock threading of the cap to create one continuous passage from the probe needle to the piercing tips 251. This continuous passage may direct fluid from the cartridges through the engagement of the containment unit and the probe needle into the patient with the application of pressure to the plunger rod. In one embodiment, cap 250) is engaged by a press-fit or similar mechanism to the containment unit.

The dual-cartridge combination product injector 200 allows for the simultaneous delivery of the contents of two separate container closure systems into one injection site. Injector 200 may provide a simple, easy-to-use method to accurately delivery two drug product solutions from separate container closure systems or vials with an easy to recognize design. One advantage of these configurations is that they provide an easier way to administer medication. Specifically, injector 200 requires less steps and needle sticks for patients to self-administer two medicines at home, reduces environmental waste, increases stability of the drug formulation and shelf life individually, reduces impact from complicated mixing interactions and medication errors, and improves access to underserved markets.

To use the system, a clinician, patient, or user may assemble the components as described above, or the components may be pre-assembled by a manufacturer or care provider. Two separate vials or containers may be inserted into barrels 241a,241b with a pierceable member of each facing branches 252 of Y-shaped conduit 250. In some examples, Y-shaped conduit 250 includes a hollow set of tubing (e.g., a polymer tubing) or stainless-steel components. The piercing tips 251 of branches 252 may penetrate the containers to provide fluid communication between the probe needle (not shown) and the interior of the containers. To deliver the drug product into the patient, the user may simply grasp the flange 220 with, for example, their index and middle fingers, and press against the main brace 211 of plunger 210 with their thumb so that the plunger rod pushes the stoppers 230a,230b and translates them through the barrels 241a,241b, causing the contents of the containers to flow through Y-shaped conduit and the probe needle into the patient's body.

In some examples, the two containers include a two-drug product combination so that two different substances may be stored separately and introduced together. For example, a first liquid may be disposed in the first container, and a second liquid may be disposed in the second container. Alternatively, a liquid may be disposed in the first container, and a lyophilized product may be disposed in the second container. In at least some examples, the first container may contain a first pharmaceutical composition or drug substance, and the second container may contain a second pharmaceutical composition or drug substance. In a further embodiment, the first pharmaceutical composition and the second composition will be mixed and delivered through one central flow path into the patient. In another embodiment, the first drug substance and the second drug substance will be mixed and delivered through one central flow path into the patient. In another embodiment, the first container and the second container comprise any combination of injectable product or pharmaceutical composition that would be beneficial to combine to provide to a patient. In another embodiment, the first container and second container comprise any combination of pharmaceutical compositions that would be beneficial to combine for an investigative use.

It is to be understood that the embodiments described herein are merely illustrative of the principles and applications of the present disclosure. For example, the number, positioning, and arrangement of cartridges may be varied and a containment unit having three or more barrels is possible. Additionally, though one example of an injector is disclosed, it will be understood that the principles disclosed herein are equally applicable to other types of injectors (e.g., syringes with staked needles, auto-injectors, or on-body (wearable) injectors, etc.).

Additionally, more than two compartments arranged in parallel may be formed.

Moreover, certain components are optional, and the disclosure contemplates various configurations and combinations of the elements disclosed herein. It is therefore to be understood that numerous modifications may be made to the illustrative embodiments and that other arrangements may be devised without departing from the spirit and scope of the present disclosure as defined by the appended claims.

It will be appreciated that the various dependent claims and the features set forth therein can be combined in different ways than presented in the initial claims. It will also be appreciated that the features described in connection with individual embodiments may be shared with others of the described embodiments.

1. A multi-cartridge injector comprising:
a plunger having a main brace and a pair of stems, each of the pair of stems having a coupler;
a pair of stoppers coupled to each coupler of the pair of stems;
a containment unit having a pair of barrels; and
a Y-shaped conduit having a pair of branches and a common shaft, the Y-shaped conduit defining a passageway that extends through the common shaft and the pair of branches, each of the pair of branches being in communication with an interior of one of the pair of barrels.

2. The multi-cartridge injector of claim 1, further comprising a cap coupled to an end of the containment unit to seal the pair of barrels.

3. The multi-cartridge injector of claim 2, wherein the cap includes a funnel portion at a first end and a luer fitting at a second end.

4. The multi-cartridge injector of claim 3, wherein the cap is coupled to the Y-shaped conduit.

5. The multi-cartridge injector of claim 3, further comprising a disposable probe needle coupleable to the luer fitting.

6. The multi-cartridge injector of claim 1, wherein the pair of barrels share a common wall.

7. The multi-cartridge injector of claim 1, further comprising a flange having a pair of apertures, each of the pair of stems passing through at least one of the pair of apertures.

8. The multi-cartridge injector of claim 7, wherein the flange and the containment unit are unitary.

9. The multi-cartridge injector of claim 1, wherein each of the pair of barrels is generally cylindrical.

10. The multi-cartridge injector of claim 1, wherein the main brace is concave.

11. The multi-cartridge injector of claim 1, wherein each of the pair of stems has a cruciform cross-sectional shape.

12. The multi-cartridge injector of claim 1, wherein each of the containment unit and the plunger is symmetrical about a longitudinal central axis.

13. A multi-cartridge injector comprising:
a containment unit having a pair of barrels; and
a Y-shaped conduit having a pair of branches and a common shaft, the Y-shaped conduit defining a passageway that extends through the common shaft and the pair of branches, each of the pair of branches being in communication with an interior of one of the pair of barrels.

14. The multi-cartridge injector of claim 13, further comprising a cap coupled to an end of the containment unit to seal the pair of barrels.

15. The multi-cartridge injector of claim 14, wherein the cap includes a funnel portion at a first end and a luer fitting at a second end.

16. The multi-cartridge injector of claim 15, wherein the cap is coupled to the Y-shaped conduit.

17. The multi-cartridge injector of claim 13, wherein the pair of barrels share a common wall.

18. The multi-cartridge injector of claim 13, further comprising a flange having a pair of apertures, each of the pair of stems passing through at least one of the pair of apertures.

19. The multi-cartridge injector of claim 13, further comprising a plunger having a main brace and a pair of stems, each of the pair of stems having a coupler.

20. The multi-cartridge injector of claim 19, further comprising a pair of stoppers coupled to each coupler of the pair of stems.
